(19) United States
(12) Patent Application Publication
(10) Pub. No.: US 2010/0311130 A1
(43) Pub. Date: Dec. 9, 2010

(54) ENZYMATIC ESTER HYDROLYSIS

(75) Inventors: Peter Jan Leonard Mario Quaedflieg, Elsloo (NL); Claudia Cusan, Aachen (DE); Bas Ritzen, Nijmegen (NL); Aylvin Jorge Angelo Athanasius Dias, Maastricht (NL)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., Heerlen (NL)

(21) Appl. No.: 12/664,452

(22) PCT Filed: Jun. 19, 2008
(86) PCT No.: PCT/EP2008/057780

§ 371 Date: Aug. 24, 2010

(30) Foreign Application Priority Data
Jun. 19, 2007 (EP) 07011961.5

(51) Int. Cl. C12P 13/02

(52) U.S. Cl. 435/129

(57) ABSTRACT

The present invention relates to a method for selectively hydrolysing a pendant ester bond formed by an unsubstituted or substituted hydrocarbon group—optionally comprising one or more heteroatoms—and a pendant carboxylate moiety, which carboxylate moiety is part of a polymer or a polymerisable compound, which polymer or polymerisable compound comprises at least one other hydrolysable group, wherein the method comprises contacting the polymer or polymerisable compound with a hydrolytic enzyme.

The invention relates to a method for selectively hydrolysing an ester in a polymer or polymerisable compound.

Polymers or polymerisable compounds, such as monomers, macromers or prepolymers, comprising a carboxylic acid group find wide-spread use, in biomedical applications. For instance, the carboxylic acid may serve as a group to which a functional compound, e.g. a biologically active agent, can be attached. However, it is often desirable or even necessary that the carboxylic acid group is protected at some stage in the preparation of a product, in order to allow a specific process step to take place efficiently and/or to avoid an undesired side reaction due to the presence of a free (i.e. unprotected) carboxylic acid group. The term free carboxylic acid group as used herein includes the carboxylic acid group as a free acid group, as an ionised group and as a complex with a counter ion other than a proton, e.g. an alkali metal ion.

Suitably, the acid is esterified with a hydrocarbon to protect the acid. However, subsequent deprotection may be a challenge, in particular in case the compound comprises one or more other hydrolysable groups, in addition to the protected carboxylic acid group.

Before being able to couple a functional group which is part of a bioactive agent to a carboxylic acid, it is generally needed to deprotect the functional group. Deprotection is in particular troublesome in case the polymer or polymerisable compound comprises one or more other hydrolysable groups, for example further ester groups.

Hydrolysable groups such as ester groups are normally hydrolysed by an acid or base in an aqueous environment. It is however known that such a hydrolysis is not selective. In some cases a selective hydrolysis is required in particular if for example a polymer or polymerisable compound comprises one or more other hydrolysable groups such as multiple ester groups in the polymer backbone. Those multiple ester groups in the polymer backbone may not be destroyed if the polymer backbone has a functional property in the final application e.g. hydrophilicity or crosslink density. Therefore there is a need for selective hydrolysis.

It is known that a selective hydrolysis of a t-butyl ester over some other ester groups can be achieved preferentially in a chemical process for example with trifluoroacetic acid (TFA) in a dry organic solvent. However, several disadvantages are associated with this process. For an efficient deprotection of the ester it is generally required to use a large excess of TFA (>10 equivalents). The highly acidic conditions required make this form of deprotection unsuitable for compounds that are not stable in strongly acidic conditions. The reaction is carried out in a dry solvent as a trace of water during the TFA-mediated deprotection would usually be sufficient to cause extensive hydrolysis of other hydrolysable groups, in particular other ester functions in the molecule. Complete or almost complete removal of TFA is laborious (and expensive) but of crucial importance, in particular in case a functional group, e.g. a functional group which is part of a biomolecule, is to be coupled to the carboxylic acid, since the presence of TFA in the coupling step may be detrimental to the attachment reaction.

In view of the above, it is a problem to selectively hydrolyse an ester if the compound also comprises other hydrolysable bonds. In particular it is a problem to rapidly and/or efficiently synthesize urethanes, thiourethanes, amides, and the like, bearing a free carboxylic acid function, if there are one or more ester functionalities or other hydrolysable bonds elsewhere in the molecule. More in particular for polymers or polymerisable compounds based on isocyanates of an amino acid, for example polymers or polymerisable compounds based on lysine diisocyanate (LDI), it is a problem to be synthesised selectively, rapidly and/or efficiently.

For instance, LDI-based urethanes can be synthesised by reaction of a lysine-diisocyanate (LDI) moiety and alcohols. For the synthesis of an LDI moiety from lysine (e.g. with phosgene or a derivative thereof) the carboxyl function needs to be protected, since otherwise the free carboxyl function would likely give cause to one or more undesired side reactions. Additionally, the free amino acid is mostly not soluble enough in the reaction medium (free amino acids usually only dissolve in aqueous solution).

It is an object of the present invention to provide a method for selectively hydrolysing an ester of a polymer or a polymerisable compound that comprises at least one other hydrolysable group.

In particular, it is an object of the present invention to overcome one or more disadvantages such as indicated above.

It has now been found possible to selectively hydrolyse a pendant ester of a polymer or a polymerisable compound that comprises at least one other hydrolysable group.

Accordingly, the present invention relates to a method for selectively hydrolysing a pendant ester formed by a hydrocarbon group and a pendant carboxylate moiety in which the pendant carboxylate moiety is part of a polymer or a polymerisable compound, that comprises at least one other hydrolysable group, wherein the method comprises contacting the polymer or polymerisable compound with a hydrolytic enzyme to catalyse the hydrolysis of the pendant ester.

By a pendant ester is meant an ester that is not in the polymer backbone or will not be in the resultant polymer backbone in a subsequent polymerisation step.

It has surprisingly been found possible to hydrolyse a pendant ester, with a high degree of selectivity over one or more other hydrolysable groups, for example other ester groups, urethane groups or urea groups, present in the backbone chain of the polymer or polymerisable compound.

It would normally be expected that the hydrolysable groups which are part of the polymer backbone or polymerisable compound, for example hydrolysable groups in a terminal position will be hydrolysed first. In the present invention it has however been found that the hydrolytic enzyme is able to selectively hydrolyse the pendant ester even despite of the fact that the hydrolytic enzyme has to overcome steric hindrance of the polymer backbone.

As used herein, the term “polymer” denotes a structure that essentially comprises a multiple repetition of units derived, actually or conceptually, from molecules of low relative molecular mass. Such polymers may include crosslinked networks, dendrimeric and hyperbranched polymers and linear polymers. Oligomers are considered a species of polymers, i.e. polymers having a relatively low number of repetitions of units derived, actually or conceptually, from molecules of low relative molecular mass.

Polymers may have a molecular weight of 200 Da or more, 400 Da or more, 800 Da or more, 1000 Da or more, 2000 Da or more, 4000 Da or more, 8000 Da or more, 10 000 Da or more, 100 000 Da or more or 1 000 000 Da or more. Polymers having a relatively low mass, e.g. of 8000 Da or less, in particular 4000 Da or less, more in particular 1000 Da or less may be referred to as oligomers.

Within the context of the present invention the term “hydrocarbon” is meant to include substituted and unsubstituted hydrocarbons, hydrocarbons with optionally one or more heteroatoms (such as S, N, O, P, halogens). Substituents may in particular be selected from —OH and halogen atoms (Br, Cl, F, I).

With a hydrolytic enzyme is meant an enzyme with the ability to catalyse the hydrolysis of a carboxylic ester to form the corresponding free carboxylic acid.

The present invention in particular relates to a method for selectively hydrolysing a pendant ester formed by a hydrocarbon group and a pendant carboxylate moiety, which pendant carboxylate moiety is part of a polymer or a polymerisable compound comprising (a) at least two polymerisable moieties and (b) at least one amino acid residue.

For example if the polymerisable compound is based on a diisocyanate of a diamino acid (such as LDI) and a hydroxyalkylacrylate, e.g. as represented by formula I below, it has been found possible to almost completely or completely hydrolyse the pendant ester functionality of the amino acid moiety, without any detectable hydrolysis of other hydrolysable groups in the polymerisable compound.

It is in particular surprising that the invention allows the selective hydrolysis of an inaccessible pendant ester in a compound such as a polymer or oligomer or a large polymerisable compound, for example compounds comprising more than one polymerisable moiety.

The method according to the present invention is particularly useful to selectively hydrolyse a pendant ester of a polymer or polymerisable compound comprising (a) at least two polymerisable moieties, (b) at least one amino acid residue of an amino acid comprising at least two amine groups of which at least two amine groups have formed a urea group, a thio-urea group, a urethane group or a thio-urethane group and (c) the hydrocarbon linked to the carboxylic acid of the amino acid.

The invention thus allows the selective hydrolysis of a pendant ester bond in a polymer or polymerisable compound obtained from commercially readily available or easily synthesisable starting compounds. For example a urethane can be prepared from a diamino acid of which the carboxylic acid function is protected with a primary alkyl ester, for example a methylester, such as L-lysine methylester.

Further, it is advantageous that a highly selective hydrolysis is achievable without needing an excess of reagents, such as an excess of an acid.

The polymer or polymerisable compound may comprise, in addition to the pendant ester formed by the hydrocarbon group and the pendant carboxylate moiety, a moiety selected from urea groups, thio-urea groups, urethane groups, thio-urethane groups, other ester groups, amide groups, glycopeptide groups, carbonate groups, sulphones and carbohydrate groups.

The method according to the present invention is more in particular useful to selectively hydrolyse a pendant ester of a polymerisable compound represented by the formula I wherein:

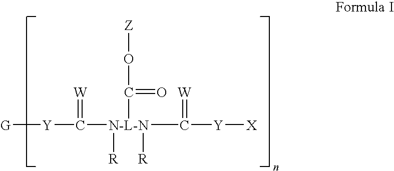

G is a residue of a polyfunctional compound having at least n functional groups or a moiety X.
each X independently represents a moiety comprising a polymerisable group.
in case that G=X, formula I represents a polymerisable compound
in case that G is different from X, formula I represents a polymer or oligomer.
each Y independently represents O, S or NR.
each W independently represents O or S.
each R independently represents hydrogen or a group selected from substituted and unsubstituted hydrocarbons which optionally contain one or more heteroatoms,
L represents a substituted or unsubstituted hydrocarbon group which optionally contains one or more heteroatoms
n is an integer having a value of at least 1 and
Z is a substituted or unsubstituted hydrocarbon group bound to the carboxylate moiety.

In principle, G is multifunctional polymer or oligomer optionally functionalised with an —OH, —NH₂, —RNH or —SH, where the group that reacts to give formula I is —OH, —NH₂, —RNH or —SH. G is preferably selected from polyesters, polythioesters, polyorthoesters, polyamides, polythioethers and polyethers.

In particular, G may be selected from polylactic acid (PLA); polyglycolide (PGA); polyanhydrides; polytrimethylenecarbonates; polyorthoesters; polydioxanones; poly-ε-caprolactones (PCL); polyurethanes; polyvinyl alcohols (PVA); polyalkylene glycols, for example polyethyleneglycol (PEG); polyalkylene oxides, preferably selected from polyethylene oxides or polypropylene oxides; polyethers; poloxamines; polyhydroxy acids; polycarbonates; polyaminocarbonates; polyvinyl pyrrolidones; polyethyl oxazolines; carboxymethyl celluloses; hydroxyalkylated celluloses, such as hydroxyethyl cellulose and methylhydroxypropyl cellulose; and natural polymers, such as polypeptides, polysaccharides and carbohydrates, such as polysucrose, hyaluranic acid, dextran and derivatives thereof, heparan sulfate, chondroitin sulfate, heparin, alginate, and proteins such as gelatin, collagen, albumin, or ovalbumin; and co-oligomers, copolymers, and blends of comprising any of these moieties.

The moiety G may be chosen based upon its biostability and/or biodegradability properties. For providing a compound or polymer or article with a high biostability, polyethers, polythioethers, aromatic polyesters, aromatic thioesters are generally particularly suitable. Preferred examples of oligomers and polymers that impart biodegradability include aliphatic polyesters, aliphatic polythioesters, aliphatic polyamides and aliphatic polypeptides.

Preferably, G is selected from polyesters, polythioesters, polyorthoesters, polyamides, polythioethers and polyethers. Good results have in particular been achieved with polyethers, in particular with a polyalkylene glycol, more in particular with polyethyleneglycol (PEG).

For a hydrophobic polymer, G may suitably be selected from hydrophobic polyethers such as polybutylene oxide or polytetramethyleneglycol (PTGL).

A polyalkylene glycol, such as PEG is advantageous in an application wherein a product may be in contact with a body fluid containing proteins, for instance blood, plasma, serum or a extracellular matrix. It may in particular show a low tendency to foul (low non-specific protein absorption) and/or have an advantageous effect on the adhesion of biological tissue. A low fouling is desirable, in order to avoid shielding of moiety Z by fouling proteins and the like.

The number average molecular weight (Mn) of the moiety G is usually at least 200 g/mol, in particular at least 500 g/mol. For an improved mechanical property, Mn preferably is at least 2000 g/mol. The number average molecular weight of the moiety G is usually up to 100 000 g/mol. Herein the number average molecular weight is as determinable by size exclusion chromatography (SEC).

The hydrocarbon group which—during hydrolysis—is cleaved from the carboxylate moiety may in principle be any substituted or unsubstituted hydrocarbon group, optionally comprising one or more heteroatoms, such as one or more heteroatoms selected from the group of N, S, O, Cl, F, Br and I. Usually, the number of hydrocarbons is 1-20, preferably 1-10, more preferably 1-6. The hydrocarbon may be linear, branched or cyclic. Most preferred are alkyl groups, because alkyl groups are highly suitable as a protective group and can suitably be removed enzymatically. The alkyl group may be an unsubstituted alkyl group or a substituted alkyl group, for example a hydroxyalkyl group.

For a high hydrolysis rate a lower alkyl group may be preferred, such as methyl, ethyl, or n-propyl. Most preferably the alkyl group is a methyl group.

For a high hydrolysis rate and/or in view of the ease of obtaining the ester, the ester is preferably a primary alkyl ester, although the invention may in principle also be employed to hydrolyse a secondary alkyl ester, for example an iso-propyl ester or a tertiary alkyl ester, for example a t-butylester.

In principle, the polymerisable moiety (such as “X”, in Formula I) in the polymerisable compound can be any moiety that allows formation of a polymer. In particular it may be chosen from moieties that are polymerisable by an addition reaction. Such type of reaction has been found easy and well-controllable. Further, it may be carried out without formation of undesired side products, such as products formed from leaving groups.

Preferably, the polymerisable moiety allows radical polymerisation. This has been found advantageous as it allows initiating a polymerisation, in the presence of a water-photo-initiator, by electromagnetic radiation, such as UV, visible light, microwave, near-IR, gamma radiation, or by electron beam instead of thermally initiating the polymerisation reaction. This allows rapid polymerisation, with no or at least a reduced risk of thermal denaturation or degradation of (parts of) the compound/the polymer. Thermal polymerisation may be employed, in particular in case no biological moiety or moieties are present that would be affected by heat. E.g. heat-polymerisation may be employed when one or more oligo-peptides and/or proteins are present of which the active sites are not affected by the high temperature, required for polymerisation at elevated temperatures.

Preferred examples of the polymerisable moiety (such as “X”, in Formula I) include groups comprising an unsaturated carbon-carbon bond—such as a C═C bond (in particular a vinyl group) or a C≡C group (in particular an acetylene group), thiol groups, epoxides, oxetanes, hydroxyl groups, ethers, thioethers, HS—, H₂N—, —COOH, HS—(C═O)— or a combination thereof, in particular a combination of thiol and C═C groups.

In particular preferred is a polymerisable moiety selected from the group consisting of an acrylate including hydroxyl(meth)acrylates; alkyl(meth)acrylates, including hydroxyl alkyl(meth)acrylates; vinylethers; alkylethers; unsaturated diesters and unsaturated diacids or salts thereof (such as fumarates); and vinylsulphones, vinylphosphates, alkenes, unsaturated esters, fumarates, maleates or combinations thereof. More preferred is a moiety selected from acrylates, methacrylates, itaconates, vinylethers, propenylethers, alkylacrylates and alkylmethacrylates. Most preferred is a moiety selected from (meth)acrylates and alkyl(meth)acrylates, especially hydroxy alkylmethacrylates and hydroxy alkylacrylates. Such moiety can be introduced in the polymer or polymerisable compound of the invention starting from readily available starting materials and show good biocompatibility, which makes these particularly useful for an in vivo or other medical application.

Good results have in particular been achieved with a polymerisable compound wherein the X-Y moiety represents hydroxyethylacrylate or hydroxyethyl methacrylate.

In further preferred embodiment, the polymerisable moiety X is represented by the formula —R₁R₂C═CH₂, wherein
R₁ is chosen from the group of substituted and unsubstituted, aliphatic, cycloaliphatic and aromatic hydrocarbon groups that optionally contain one or more moieties selected from the group consisting of ester moieties, ether moieties, thioester moieties, thioether moieties, urethane moieties, thiourethane moieties, amide moieties and other moieties comprising one or more heteroatoms, in particular one or more heteroatoms selected from S, O, P and N. R₁ may be linear or branched. In particular R₁ may comprise 2-20 carbon atoms, more in particular it may be a substituted or unsubstituted C₁ to C₂₀ alkylene; more in particular a substituted or unsubstituted C₂ to C₁₄ alkylene.
R₂ is chosen from the group of hydrogen and substituted and unsubstituted alkyl groups, which alkyl groups optionally contain one or more heteroatoms, in particular one or more heteroatoms selected from P, S, O and N. R₂ may be linear or branched. In particular, R₂ may be hydrogen or a substituted or unsubstituted C₁ to C₆ alkyl, in particular a substituted or unsubstituted C₁ to C₃ alkyl.

The carboxylate group which forms the ester with the hydrocarbon that is cleaved during hydrolysis is usually based on an amino acid. In the polymer or polymerisable compound the amino acid residue (“L” in formula I) is a substituted or unsubstituted hydrocarbon, which may contain heteroatoms, such as N, S, P and/or O.

The amino acid residue L may be based on a D-isomer or an L-isomer of an amino acid. Preferably, L is C1-C20 hydrocarbon, more preferably, L is a linear or branched C1-C20 alkylene, even more preferably C2-C12 alkylene, most preferably C3-C8 alkylene, wherein the alkylene may be unsubstituted or substituted and/or optionally contains one or more heteroatoms. In view of desirable hydrophilic properties, the number of carbons is preferably relatively low, such as 8 or less.

Particularly preferred are residues based on an amino acid selected from the group of lysine, arginine, asparagine, ornithine, glutamine, hydroxylysine, methylated lysine and diaminobutanoic acid residues.

More in particular the present invention relates to a method wherein the polymerisable compound is represented by formula I in which
G=X;
each X-Y moiety represents a moiety comprising hydroxyethylacrylate or hydroxyethylmethacrylate;
each R independently represents hydrogen.
L represents an amino acid residue
n is an integer having a value of 1
W is O and
Z is a methyl, ethyl or n-propyl group bound to the carboxylate moiety.

In particular in case the polymer or polymerisable compound is intended to be used in a medical application, more in particular in case it is intended to be used in vivo, it is preferred that the amino acid residue is based upon a natural amino acid. This is in particular desired in case the compound or polymer is biodegradable. In view thereof, preferred amino acid residues are residues of lysine, hydroxylysine, methylated lysine, arginine, asparagine and glutamine in the L- or D- or D, L-configuration or any mixture of D or L-isomers. Preferably the amino acid residues are in the L-configuration. Good results have in particular been achieved with L-lysine.

If desired, a functional moiety may be attached to the carboxylic acid formed after selective hydrolysis. In principle any moiety may be attached that can be attached to a carboxylic acid. The moiety may be attached based on a method known in the art.

In particular the functional moiety may be an active agent selected from pharmaceuticals, stabilisers, antithrombotic moieties, moieties increasing hydrophilicity and moieties increasing hydrophobicity.

The active agent may for instance be selected from cell signalling moieties, moieties capable of improving cell adhesion to the compound/polymer/article, moieties capable of controlling cell growth (such as stimulation or suppression of proliferation), anti-thrombotic moieties, moieties capable of improving wound healing, moieties capable of influencing the nervous system, moieties having selective affinity for specific tissue or cell types and antimicrobial moieties. The moiety may exert an activity when bound to the remainder of the compound/polymer/article and/or upon release therefrom.

Examples of active agents that may be coupled include perfluoralkanes (increasing hydrophobicity); polyalkylene oxides, such as polyethylene oxide and polypropylene oxide (increasing hydrophilicity and/or for reduced fouling); polyoxazolines; amino acids; peptides, including cyclic peptides, oligopeptides, polypeptides, glycopeptides and proteins, including glycoproteins; nucleotides, including mononucleotides, oligonucleotides and polynucleotides; and carbohydrates.

For instance, an amino acid may be linked for stimulating wound healing (arginine, glutamine) or to modulate the functioning of the nervous system (asparagine).

In a preferred embodiment, the active moiety is a peptide, more preferably an oligopeptide. For instance peptides can be epitopes which may enhance or suppress biological response for example cellular growth proliferation or enhanced cell adhesion. In the case that for example enhanced antibody binding is required epitopes are the most obvious choice.

Examples of active peptides include the peptides listed in the table I.

| | |
|---|---|
| Peptide | suggested function |

NH2 to COOH direction

| | |
|---|---|
| RGD, GRGDS, RGDS | Enhance bone and/or cartilage tissue formation; Regulate |
| | neurite outgrowth; Promote myoblast adhesion, proliferation |
| | and/or differentiation; Enhance endothelial cell adhesion and/or |
| | proliferation |
| PHSRN | Synergistic peptide for RGD |
| KQAGDV | Smooth muscle cell adhesion |
| YIGSR | Cell adhesion |
| REDV | Endothelial cell adhesion |
| GTPGPQGIAGQRGVV | Cell adhesion (osteoblasts) |
| (P-15) | |
| PDGEA | Cell adhesion (osteoblasts) |
| IKVAV | Neurite extension |
| RNIAEIIKDI | Neurite extension |
| KHIFSDDSSE | Astrocyte adhesion |
| VPGIG | Enhance elastic modulus of artificial extra-cellular-matrix |
| | (ECM) |
| FHRRIKA | Improve osteoblastic mineralization |
| KRSR | Osteoblast adhesion |
| KFAKLAARLYRKA | Enhance neurite extension |
| KHKGRDVILKKDVR | Enhance neurite extension |
| YKKIIKKL | Enhance neurite extension |
| NSPVNSKIPKACCVPT | Osteoinduction |
| ELSAI | |
| APGL | Collagenase mediated degradation |
| VRN | Plasmin mediated degradation |
| AAAAAAAAA | Elastase mediated degradation |
| Ac-GCRDGPQ- | Encourage cell-mediated proteolytic degradation, |
| GIWGQDRCG | remodeling and/or bone regeneration (with RGD and BMP- |
| | 2 presentation in vivo) |
| angiotensin | vasoconstriction, increased blood pressure, release of |
| | aldosterone from the adrenal cortex. |
| HSWRHFHTLGGG | Binds to monocyte chemo attractant protein (MCP-1) |

A preferred example of a cyclic peptide is gramacidin S, which is an antimicrobial.

Further examples of suitable peptides in particular include: vascular endothelial growth factor (VEGF), transforming growth factor B (TGF-B), basic fibroblast growth factor (bFGF), epidermal growth factor (EGF), osteogenic protein (OP), monocyte chemoattractant protein (MCP 1), tumour necrosis factor (TNF), Examples of proteins which may in particular form part of a compound of the invention include growth factors, chemokines, cytokines, extracellular matrix proteins, glycosaminoglycans, angiopoetins, ephrins and antibodies.

A preferred carbohydrate is heparin, which is antithrombotic.

A nucleotide may in particular be selected from therapeutic nucleotides, such as nucleotides for gene therapy and nucleotides that are capable of binding to cellular or viral proteins, preferably with a high selectivity and/or affinity.

Preferred nucleotides include aptamers. Examples of both DNA and RNA based aptamers are mentioned in Nimjee et. al. Annu. Rev. Med. 2005, 56, 555-583. The RNA ligand TAR (Trans activation response), which binds to viral TAT proteins or cellular protein cyclin T1 to inhibit HIV replication, is an example of an aptamer. Further, preferred nucleotides include VA-RNA and transcription factor E2F, which regulates cellular proliferation.

The hydrolytic enzymes suitable for use in accordance with the present invention can be immobilized, in particular loaded on a support such as, for example, an acrylic support, or used in their unsupported, i.e., free form. Suitable immobilisation techniques are generally known in the art.

The hydrolytic enzyme may in particular be chosen from enzymes classified as hydrolases acting on ester bonds (E.C. 3.1.) or hydrolases acting on peptide bonds (E.C. 3.4.). Preferably the hydrolytic enzyme is chosen from enzymes classified as carboxylic ester hydrolases (E.C. 3.1.1.), serine endopeptidases (E.C. 3.4.21.), aminopeptidases (E.C. 3.4.11.), cysteine endopeptidases (E.C. 3.4.22.), aspartic endopeptidases (E.C. 3.4.23.), metalloendopeptidase (E.C. 3.4.24.) or endopeptidases of unknown catalytic mechanism (E.C. 3.4.99.). Most preferably the hydrolytic enzyme is chosen from the group of protease enzymes classified as papain (E.C. 3.4.22.2.) or subtilisins (E.C. 3.4.21.62.) or the hydrolytic enzyme is chosen from the group of carboxylic ester hydrolases (E.C. 3.1.1.3) chosen from lipozyme, for example Lipozyme RM available from Novozyme, lipase from Rhizomucor Miehei, for example lipase R. Miehei available from Novozyme, lipase B from Candida Antarctica, for example CaIB available from Novozyme, lipase from Penicillium Camembertii for example lipase G “Amano”50 available from Amano Enzyme Inc or lilipase available from Nagase.

The hydrolytic enzyme may be obtained or derived from any organism, in particular from an animal, a plant, a bacterium, a mould, yeast or a fungus. When referred to an enzyme from a particular source, recombinant enzymes originating from a first organism, but actually produced in a (genetically modified) second organism, are specifically meant to be included as enzymes from that first organism.

In particular good results have been achieved with a peptidase, especially with endopeptidase, more preferably with papain or subtilisin in order to selectively hydrolyse a primary alkyl ester, more in particular to hydrolyse a methyl ester. Particularly preferred is Subtilisin Carlsberg®.

It has surprisingly been found that the reaction can efficiently be carried out by using Alcalase®, available from Novozymes (Bagsvaerd, Denmark). Alcalase® is a cheap and industrially available proteolytic enzyme mixture produced by Bacillus licheniformis (containing Subtilisin Carlsberg® as a major enzyme component).

In an embodiment of the invention, a suitable hydrolytic enzyme for selective hydrolysis may be selected from the group of the following commercially available products, and functional analogues of such enzymes.

Proteinase-K is available from New England Biolabs, Ipswich (Mass.), USA).

Novo Nordisk Biochem North America Inc (Franklinton N.C., USA) offers Protease Bacillus species (Esperase 6.0 T; Savinase 6.0 T), Protease Bacillus subtilis (Neutrase 1.5 MG), Protease Bacillus licheniformis (Alcalase 3.0 T).

Amano International Enzyme Co (Troy, Va., USA) offers Protease Bacillus subtilis (Proleather; Protease N) and Protease Aspergillus oryzae (Prozyme 6).

The amount of enzyme present or used in the process is difficult to determine in absolute terms (e.g. grams), as its purity is often low and a proportion may be in an inactive, or partially active, state. More relevant parameters are the activity of the enzyme preparation and the activities of any contaminating enzymes. These activities are usually measured in terms of the activity unit (U) which is defined as the amount which will catalyse the transformation of 1 micromole of the substrate per minute under standard conditions. Typically, this represents 10⁻⁶-10⁻¹¹ Kg for pure enzymes and 10⁻⁴-10⁻⁷ Kg for industrial enzyme preparations. The amount of hydrolytic enzyme per gram of polymer or polymerisable compound in principle is not critical and may for instance depend on the reactivity of the pendant ester moiety and on the enzyme cost price. A typical amount of enzyme ranges from 0.01-1000 U per gram of polymer of polymerisable compound. Preferably 0.1-100 U/g are used and most preferably 1-10 U/g.

The hydrolysis can in general be carried out under mild and/or environmentally friendly conditions. For instance, no highly acidic or alkaline conditions are required which would hydrolyse the other hydrolysable groups present in the polymer or polymerisable compound. Usually, the hydrolysis may be carried out at an approximately neutral pH, a slightly alkaline or a slightly acidic pH, for example at a pH between 4-10, in particular at a pH between 5-9±2 pH units.

In principle also a more alkaline or acidic pH may be used, in particular if the enzyme shows sufficiently selective activity to allow selective hydrolysis. A favourable pH may be chosen based on a known or empirically determinable activity curve for the enzyme as a function of pH and the information disclosed herein.

The apparent pH (the pH measured with a calibrated pH electrode in the reaction medium, at 25° C.) is usually at least 5 in order to avoid undesired acidic hydrolysis of one or more hydrolysable groups, and/or for a high selectivity of the enzyme for the ester that is desired to be hydrolysed. In particular, the apparent pH may be at least 6, more in particular at least 6.5. The apparent pH usually is up to 9, in order to avoid undesired alkaline hydrolysis of one or more hydrolysable groups, and/or for a high selectivity of the enzyme for the ester that is desired to be hydrolysed. In particular, the apparent pH may be up to 8, more in particular up to 7.5.

In particular for Subtilisin Calsberg®, such as in Alcalase®, an apparent pH in the range of 5-9 may be chosen, a pH of 6.5-7.5 being particularly preferred for a highly selective hydrolysis of the desired ester and/or a high hydrolysis rate.

The method in accordance with the invention is further advantageous in that no special precautions are needed to avoid the presence of water, as is important when using an acid such as TFA to selectively remove a hydrocarbon group from the carboxylic acid. Advantageously, the hydrolysis can be carried out in water or in a mixture of water and a water-miscible organic solvent. The water-miscible organic solvent may be used to improve the solubility of a particular substrate.

One or more organic solvents that can be used are for example chosen from solvents that are fully dissolvable in or miscible with water at molecular level, especially organic solvents. In particular, at least one organic solvent may be selected from the group of lower alcohols, for example methanol, ethanol, propanol, butanol, pentanol and hexanol. The alcohol may be a primary, secondary or tertiary alcohol. Particularly preferred are tertiary alcohols, such as t-butanol or t-amylalcohol.

If present, the weight to weight ratio of total organic solvent(s) to water usually is at least 1:99, in particular at least 5:95, at least 10:90, at least 80:20 or at least 25:75. In principle, the ratio may be up to 99:1 or more, up to 90:10, up to 80:20 or up to 60:40. Usually, it is up to 50:50, preferably up to 40:60, up to 35:65 or up to 30:70. Dependent on the solubility of the substrate H2O is present in an amount of 1 wt % preferably >5 wt % most preferably >10 wt %.

The temperature of the enzymatic hydrolysis reaction can usually be chosen within wide limits, taken into account factors such as the activity of the enzyme as a function of temperature, the stability of the enzyme at a specific temperature and the tendency of one or more hydrolysable groups other than the ester of which hydrolysis is desired to become hydrolysed at a specific temperature. Usually, the temperature is at least 0° C., in particular at least 10° C., more preferably at least 15° C. Usually, the temperature is up to 80° C. more preferably up to 60° C.

In particular, when Subtilisin Carlsberg®, is used good results have been achieved at a temperature in the range of 20° C. to 40° C.

The invention will now be illustrated by the following examples without being limited thereto.

ABBREVIATIONS

| | |
|---|---|
| CH₃CN | acetonitrile |
| d | doublet |
| dd | double doublet |
| DCM | dichloromethane |
| DIPEA | N,N-Diisopropylethylamine |
| DMEA | dimethylamino ethanol |
| DMSO | dimethylsulfoxide |
| EDC•HCl | N-(3-Dimethylaminopropyl)-N'- |
| | ethylcarbodiimide hydrochloride |
| EtOAc | Ethyl acetate |
| Fmoc-Glu-Gly-Phe-NH₂ | N-α-(9-fluorenylmethyloxycarbonyl)-L- |
| | glutamyl-L-glycyl-L-phenylalanine |
| | amide |
| Fmoc-Glu-(OMe)-Gly-Phe-NH₂ | N-α-(9-fluorenylmethyloxycarbonyl)-L- |
| | glutamyl-γ-methyl-ester-L-glycyl-L- |
| | phenylalanine amide |
| Fmoc-Glu-(OBu^{t})-Gly-Phe-NH₂ | N-α-(9-fluorenylmethyloxycarbonyl)-L- |
| | glutamyl-γ-tert-Butyl-ester-L-glycyl-L- |
| | phenylalanine amide |
| Fmoc-Glu-(OBu^{t}) | N-α-(9-fluorenylmethyloxycarbonyl)- |
| | L-glutamic acid-γ-tert-Butyl ester |
| Gly-Phe-NH₂•HCl | L-glycyl-L-phenylalanine hydrochloride |
| | salt |
| H₂O | water |
| HOAt | 1-Hydroxy-7-azabenzotriazole |
| m | multiplet |
| MTBE | tert-Butylmethylether |
| MeOH | Methanol |
| MSA | Methane sulfonic acid |
| NaHCO₃ | sodium hydrogencarbonate |
| Na₂SO₄ | sodium sulfate |
| NMR | nuclear magnetic resonance |
| s | singlet |
| t | triplet |
| TFA | trifluoroacetic acid |
| THF | tetrahydrofuran |
| TLC | Thin Layer Chromatography |

### EXAMPLE 1

### Synthesis of Me-LDI-(HEA)₂ and Me-LDI-(HEMA)₂

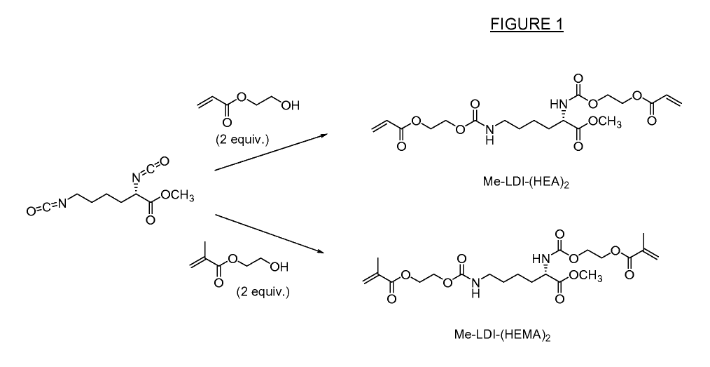

N_{α},N^{ε}-di-(2-acryloxy-ethoxycarbonyl)-L-lysine methylester (Me-LDI-(HEA)₂) and N_{α},N^{ε}-di-(2-methacryloxy-ethoxycarbonyl)-L-lysine methylester (Me-LDI-(HEMA)₂) were prepared as shown in FIG. 1.

2-Hydroxyethylacrylate (HEA, 502 mmol) or 2-hydroxyethyl-methacrylate (HEMA, 502 mmol), respectively, was added dropwise to L-lysine-diisocyanate methylester (251 mmol), tin-(II)-ethylhexanoate (0.120 g) and Irganox 1035 (150 mg) under dry air at controlled temperature (<5° C.). The reaction mixture was stirred at 40° C. for 18 h. During this time, the IR NCO vibrational stretch at v=2260 cm⁻¹ had disappeared. The solvent was evaporated in vacuo to give the product as an oil.

Me-LDI-(HEA)₂: ¹H NMR (CDCl₃, 300 MHz) δ 6.43 (dd, J=1.6 and 17.1 Hz, 2H), 6.13 (dd, J=10.3 and 17.1 Hz, 2H), 5.85 (dd, J=1.6 and 10.3 Hz, 2H), 5.39 (bs, 1H), 4.88 (bs, 1H), 4.41-4.19 (m, 9H), 3.43 (s, 3H), 3.17 (q, J=6.1 Hz, 2H), 1.90-1.78 (m, 1H), 1.76-1.60 (m, 1H), 1.57-1.45 (m, 2H), 1.42-1.27 (m, 2H). Me-LDI-(HEMA)₂: ¹H NMR (CDCl₃, 300 MHz) δ 6.13-6.10 (m, 2H), 5.57 (q, J=1.5 Hz, 2H), 5.36 (d, J=8.0 Hz, 1H), 4.85 (bs, 1H), 4.35-4.27 (m, 9H), 3.73 (s, 3H), 3.16 (q, J=6.4 Hz, 2H), 1.93 (s, 6H), 1.88-1.76 (m, 1H), 1.74-1.61 (m, 1H), 1.55-1.44 (m, 2H), 1.42-1.30 (m, 2H).

### EXAMPLE 2

### Hydrolysis of Me-LDI-(HEA)₂ to LDI-(HEA)₂ and of Me-LDI-(HEMA)₂ to LDI-(HEMA)₂ using alcalase

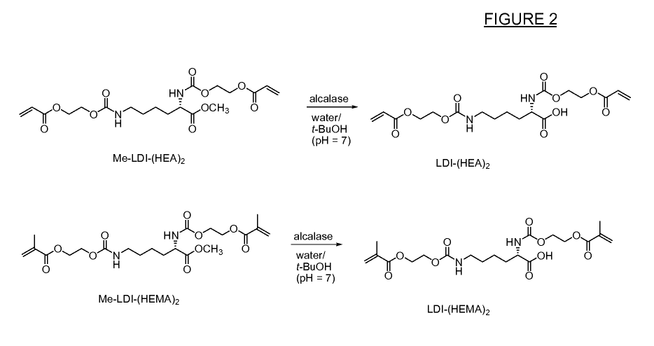

The methyl ester of Me-LDI-(HEA)₂ respectively of Me-LDI-(HEMA)₂ was selectively hydrolysed as shown in FIG. 2.

To a solution of NaH₂PO₄ (0.1 g) and NaHCO₃ (0.1 g) in 70 mL of H₂O 800 mg of liquid alcalase (from Novozymes, type 2.4 L FG, 2.4 AU/g, batch no. PMN 05076) was added and the pH was adjusted to 7.0 by the addition of NaH₂PO₄ or NaHCO₃. To this mixture a solution of 2.0 g of the methyl ester in 30 mL t-butanol was added. The reaction mixture was stirred for 4 h at ambient temperature, while the pH was kept at 7.0 by the addition of NaH₂PO₄ or NaHCO₃. After adjusting the pH to 6 by adding NaH₂PO₄, the t-butanol was evaporated in vacuo. After raising the pH to 7.0 by adding NaHCO₃, the aqueous layer was washed with dichloromethane (3×30 mL). The aqueous phase was acidified to pH 1.5 with 2 M aq. HCl and extracted with dichloromethane (3×30 mL). The combined extracts were dried (Na₂SO₄) and concentrated in vacuo giving the desired carboxylic acids in 95% yield.

The conversion during the hydrolysis reaction of Me-LDI-(HEA)₂ to LDI-(HEA)₂ was monitored by HPLC analysis using an HP1090 Liquid Chromatograph and a Prevail C₁₈ column from Alltech (250 mm×4.6 mm, particle size 5μ) operated at 40° C. UV detection was performed at 210 nm using a UVIS 204 Linear spectrometer. The flow was 1 mL/min and the injection volume 5 μL. A gradient was used, combining eluent A (10 mM aqueous H₃PO₄) and eluent B (acetonitrile). Gradient program: 0-5 min 0.5% B, 5-10 min linear gradient to 50% B, 10-16.7 min linear gradient to 90% B, 19.1 min back to 0.5% B and stop at 25 min. Retention times: Me-LDI-(HEA)₂ 14.6 min, LDI-(HEA)₂ 13.9 min. The methylester hydrolysis of Me-LDI-(HEA)₂ was found to be complete after 4 h, whereas no detectable hydrolysis products resulting from an undesired hydrolysis or other hydrolysable bonds were detected. This indicates that a high selectivity (up to 100%) for the hydrolysis of the pendant alkyl ester over the hydrolysis of another hydrolysable bond, in particular a urethane bond or acrylic ester bond is feasible.

NMR data: LDI-(HEA)₂: ¹H NMR (DMSO-d₆, 300 MHz) δ 12.83-12.32 (bs, 1H), 7.53 (d, J=7.6 Hz, 1H), 7.22 (t, J=5.0 Hz, 1H), 6.34 (d, J=17.2 Hz, 2H), 6.18 (dd, J=17.2 and 10.1 Hz, 2H), 5.96 (d, J=10.1 Hz, 2H), 4.31-4.23 (m, 4H), 4.22-4.13 (m, 4H), 3.91-3.78 (m, 1H), 2.98-2.87 (m, 2H), 1.70-1.46 (m, 2H), 1.42-1.20 (m, 4H). LDI-(HEMA)₂: ¹H NMR (CDCl₃, 300 MHz) δ 6.13 (s, 2H), 5.59 (s, 2H), 5.57-5.52 (m, 1H), 5.00-4.91 (m, 1H), 4.37-4.25 (m, 9H), 3.18 (q, J=6.5 Hz, 2H), 1.94 (s, 6H), 1.93-1.68 (m, 2H), 1.58-1.33 (m, 4H).

### EXAMPLE 3

### Hydrolysis Me-LDI-(4-pentene)₂ to LDI-(4-pentene)₂ using alcalase

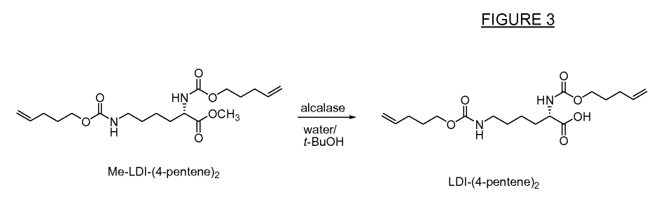

The methyl ester of N_{α},N^{ε}-di-(4-penten-1-oxycarbonyl)-L-lysine methylester (Me-LDI-(4-pentene)₂) was selectively hydrolysed as shown in FIG. 3.

To a solution of NaH₂PO₄ (0.1 g) and NaHCO₃ (0.1 g) in 70 mL of H₂O was added 1.6 g of liquid alcalase (from Novozymes, 2.4 L FG, 2.4 AU/g, batch no. PMN 05076) and the pH was adjusted to 7-8 by the addition of NaH₂PO₄ or NaHCO₃. To this mixture a solution of 4.0 g of Me-LDI-(4-pentene)₂ (10.4 mmol) in 30 mL t-butanol was added. The reaction was stirred for 4 h at ambient temperature, while the pH was kept between 7 and 8. After this time, TLC (using ethyl acetate as the eluent) had indicated complete methyl ester hydrolysis. The pH was adjusted to 6 by adding 2 N aqueous HCl and the t-butanol was evaporated in vacuo. The resulting aqueous phase was centrifuged for 5 min at 3000 rpm to spin down the enzyme. The enzyme residue was separated from the aqueous supernatant and resuspended in a mixture of MeOH (40 mL) and EtOH (40 mL) and centrifuged once more during 5 min at 3000 rpm. The alcoholic supernatant was evaporated to a residue and, after addition of the aqueous supernatant; the pH was raised to 7.5 by addition of 2 M aqueous NaOH. The resulting aqueous phase was washed with ethyl acetate (50 mL), acidified to pH=1.5 using 2 M aqueous HCl and extracted with ethyl acetate (5×70 mL). The combined organic extracts were dried (Na₂SO₄) and concentrated in vacuo giving the desired carboxylic acid in 95% yield. ¹H NMR (CDCl₃, 300 MHz) δ 6.03 (bs, 1H), 5.86-5.70 (m, 2H), 5.65 (d, J=8.0 Hz, 1H), 5.09-4.92 (m, 4H), 4.31 (bs, 1H), 4.13-3.98 (m, 4H), 3.95-3.84 (m, 1H), 3.18-3.08 (m, 2H), 2.14-2.01 (m, 4H), 1.76-1.64 (m, 4H), 1.55-1.44 (m, 6H).

### EXAMPLE 4

### Hydrolysis of Me-LDI-(HEMA)₂ to LDI-(HEMA)₂ using papain

To a solution of Me-LDI-(HEMA)₂ (55 mg, 0.11 μmol) in 0.44 mL DMF and 3.77 mL Hepes buffer (pH=8.2) was added a freshly prepared solution of 22 mg of papain (from Merck, from Carica papaya, 30000 USP-U/mg, art.7144, batch no. 333 F677044) in 0.22 mL of distilled water. The reaction mixture was stirred for 6 h at ambient temperature and then analyzed using the HPLC method as described in Example 2. This indicated that the methyl ester had been completely hydrolysed and no detectable side reactions had occurred.

### EXAMPLE 5

### Hydrolysis of Me-LDI-(HEMA)₂ to LDI-(HEMA)₂ using Lilipase

To a solution of Me-LDI-(HEMA)₂ (55 mg, 0.11 μmol) in 2.5 mL tert-butanol and 22.5 mL potassium phosphate buffer (50 mM, pH=7.5), 40 mg of Lilipase A-10FG (from Nagase, from Rhizophus Japanicus, batch n. 2535192) was added. The reaction mixture was stirred for 6 hours and then analyzed using HPLC method as described in Example 2. This indicated that 73% of methyl ester had been hydrolyzed and not detectable side reaction had occurred.

### EXAMPLE 6

### Hydrolysis of Et-LDI-(4-pentene)₂ to LDI-(4-pentene)₂ using CaIB

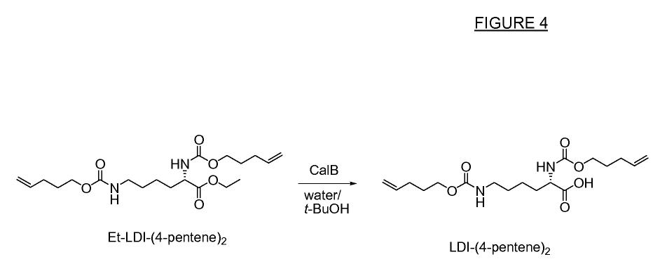

The ethyl ester of N_{α},N^{ε}-di-(4-penten-1-oxycarbonyl)-L-lysine ethyl ester (Et-LDI-(4-pentene)₂) was selectively hydrolysed as shown in FIG. 4.

To a solution of 2.0 g of Et-LDI-(4-pentene)₂ (5.0 mmol) in 10 mL t-butanol, 90 mL of phosphate buffer (pH=7.4) was added. 1 g of CaIB (from Novozyme, lipase Novozyme 435 from Candida Antarctica, batch n. LC200204) was added and the reaction mixture was stirred for 6 h at ambient temperature, while the pH was kept between 7.2 and 7.5. The mixture was analyzed using HPLC method as described in Example 2. This indicated that ethyl ester had been completely hydrolyzed and not detectable side reaction had occurred.

### EXAMPLE 7

### Synthesis of Fmoc-Glu-(OMe)-Gly-Phe-NH₂

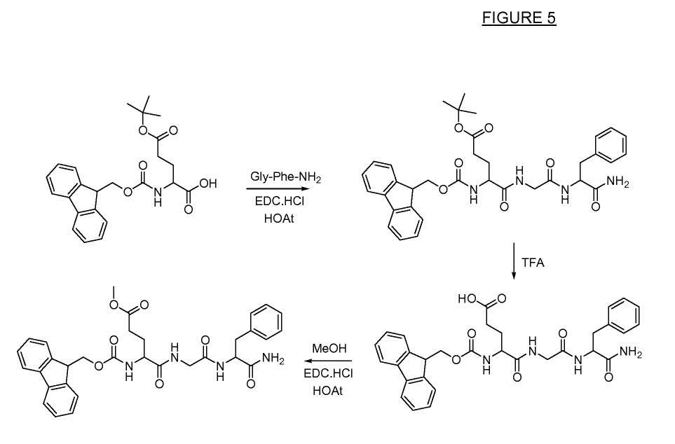

The synthesis of Fmoc-Glu-(OMe)-Gly-Phe-NH₂ was performed by following FIG. 5.

First Fmoc-Glu-(OBu^{t})-Gly-Phe-NH₂ was prepared by chemical coupling of Fmoc-Glu-(OBu^{t}) and Gly-Phe-NH₂.HCl, both commercially available; thereafter the tert-Butyl ester protecting group was cleaved using TFA and replaced with methyl ester group.

To a stirred solution of 1 g (2.25 mmol) of Fmoc-Glu-(OBu^{t}) in 60 mL DCM, 0.43 g of EDC.HCl (2.25 mmol), 0.30 g of HOAt (2.25 mmol) and 0.78 mL of DIPEA (4.50 mmol) were added at 0° C. 0.58 g of Gly-Phe-NH₂.HCl was added slowly and the mixture was stirred at room temperature overnight.

The reaction was followed by TLC using EtOAc:MeOH 9:1 as eluent

DCM was evaporated in vacuo and the residue was dissolved in EtOAc (150 mL). The organic solution was washed with NaHCO₃ aqueous saturated solution (150 mL×2), brine (150 mL×2) and distilled water (150 mL×1).

The organic layer was dried using Na₂SO₄ and the solvent was removed in vacuo, giving a white solid (1.27 g, 2.03 mmol, 90% yield).

¹HNMR (DMSO, 300 MHz): δ 8.10 (t, J=5.5 Hz, 1H), 7.98 (d, J=8.4 Hz, 1H), 7.88 (d, J=7.4 Hz, 2H), 7.71 (t, J=7.3 Hz, 2H), 7.61 (d, J=7.8 Hz, 1H), 7.41 (t, J=7.3 Hz, 2H), 7.31 (t, J=7.4 Hz, 2H), (7.26-7.05, m, 7H), 4.46-4.37 (m, 1H), 4.36-4.17 (m, 3H), 4.05-3.95 (m, 1H), 3.74 (dd, J=5.9, 16.7 Hz, 1H), 3.63 (dd, J=5.2, 16.7 Hz, 1H), 3.03 (dd, J=4.6, 13.7 Hz, 1H), 2.78 (dd, J=9.4, 13.7 Hz, 1H), 2.24 (t, J=7.8 Hz, 2H), 1.96-1.84 (m, 1H), 1.82-1.67 (m, 1H), 1.39 (s, 9H).

Next the tert-Butyl ester protecting group was cleaved using TFA.

To 1 g of Fmoc-Glu-(OBu^{t})-Gly-Phe-NH₂ (1.60 mmol), a mixture of 20 mL TFA and 0.1 mL of distilled H₂O was added dropwise. The mixture was stirred at room temperature and followed by TLC using EtOAc:MeOH 9:1 as eluent. After 3 hours, the acidic solution was concentrated in vacuo, obtaining a white solid (0.87 g, 1.52 mmol, 95% yield).

¹HNMR (DMSO, 300 MHz): δ 8.10 (t, J=5.4 Hz, 1H), 7.93 (d, J=8.3 Hz, 1H), 7.88 (d, J=7.4 Hz, 2H), 7.71 (t, J=6.8 Hz, 2H), 7.62 (d, J=7.8 Hz, 1H), 7.41 (t, J=6.8 Hz, 2H), 7.31 (t, J=7.4 Hz, 2H), 7.26-7.05 (m, 7H), 4.46-4.36 (m, 1H), 4.34-4.15 (m, 3H), 4.06-3.94 (m, 1H), 3.72 (dd, J=5.8, 16.7 Hz, 1H), 3.60 (dd, J=5.2, 16.7 Hz, 2H), 3.03 (dd, J=4.6, 13.8 Hz, 1H), 2.77 (dd, J=9.5, 13.8 Hz, 1H), 2.27-2.21 (m, 2H), 1.99-1.85 (m, 1H), 1.83-1.69 (m, 1H).

Next_Fmoc-Glu-(OMe)-Gly-Phe-NH₂ was synthesised.

To 0.5 g (0.87 mmol) of Fmoc-Glu-(OBu^{t}) dissolved in 30 mL MeOH and 30 mL CH₃CN, 0.17 g of EDC.HCl (0.87 mmol), 0.12 g of HOAt (0.87 mmol) and 0.15 mL of DIPEA (0.87 mmol) were added at 0° C. The mixture was stirred at room temperature overnight and followed by TLC using EtOAc:MeOH 9:1 as eluent.

The solvent was evaporated in vacuo and the residue was solved in EtOAc (100 mL). The organic solution was washed with NaHCO₃ aqueous saturated solution (100 mL×2), brine (100 mL×2) and distilled water (100 mL×1).

The organic layer was dried using Na₂SO₄ and the solved was removed in vacuo, giving a white solid (0.48 g, 0.81 mmol, 95% yield).

¹HNMR (DMSO, 300 MHz): δ 8.10 (t, J=5.5 Hz, 1H), 7.95 (d, J=8.4 Hz, 1H), 7.88 (d, J=7.4 Hz, 2H), 7.71 (t, J=6.5 Hz, 2H), 7.61 (d, J=7.8 Hz, 1H), 7.41 (t, J=7.4 Hz, 2H), 7.31 (t, J=7.4 Hz, 2H), 7.27-7.05 (m, 7H), 4.45-4.36 (m, 1H), 4.34-4.17 (m, 3H), 4.06-3.95 (m, 1H), 3.72 (dd, J=5.8, 16.6 Hz, 1H), 3.65-3.59 (m, 1H), 3.58 (s, 3H), 3.02 (dd, J=4.8, 13.8 Hz, 1H), 2.77 (dd, J=9.5, 13.8 Hz, 1H), 2.35 (t, J=7.8 Hz, 2H), 2.00-1.87 (m, 1H), 1.86-1.72 (m, 1H).

### EXAMPLE 8

### Enzymatic hydrolysis of methyl ester of Fmoc-Glu-(OMe)-Gly-Phe-NH₂ using Lipase G “Amano” 50

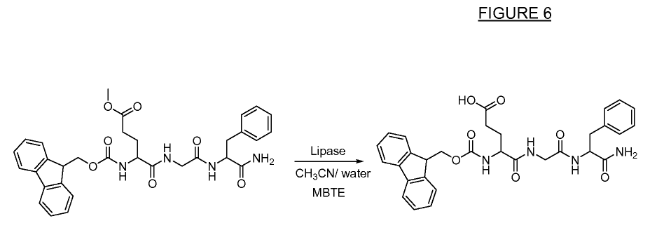

The methyl ester of Fmoc-Glu-(OMe)-Gly-Phe-NH₂ was selectively hydrolyzed as shown in FIG. 6.

100 mg of Fmoc-Glu-(OMe)-Gly-Phe-NH₂ (0.17 mmol) was dissolved in 2 mL of a mixture CH₃CN:MTBE:phosphate buffer 50 mM pH=7.5 (40:10:50). 100 mg of Lipase G “Amano”50 (from Penicillium camembertii, beach n. LGDD0352509, ≧50,000 u/g, from Amano Enzyme Inc.) was added and the reaction mixture was shaken overnight at 37° C. 100 μL sample was taken, diluted with 1 mL MeOH, filtered over syringe filter (Agilent Technologies, membrane in regenerated cellulose, 0.45 μm pore size, 13 mm diameter) and analyzed by HPLC analysis using an HP1090 Liquid Chromatograph, with an Inertsil ODS-3 (150 mm length, 4.6 mm internal diameter) column at 40° C. UV detection was performed at 220 nm using a UVVIS 204 Linear spectrometer. The gradient program was: 0-25 min linear gradient ramp from 5% to 98% buffer B and from 25.1 min to 30 min back to 5% buffer B (buffer A: 0.5 mL/L MSA in H₂O, buffer B: 0.5 mL/L MSA in CH₃CN). The flow was 1 mL/min from 0-25.1 min and 2 mL/min from 25.2-29.8 min, then back to 1 mL/min until stop at 30 min. Injection volumes were 20 μL.

The product was identified by comparison of retention time of synthesized standard.

The conversions recorded after 20 h and 48 h were 5% and 11% area percentage respectively.

$$Area perc . = ( \frac{area Fmoc \text{-} Glu \text{-} Gly \text{-} Phe \text{-} {NH}_{2}}{\begin{matrix}area Fmoc \text{-} Glu \text{-} Gly \text{-} Phe \text{-} {NH}_{2} + \\ area Fmoc \text{-} Glu \text{-} ( OMe ) \text{-} Gly \text{-} Phe \text{-} {NH}_{2}\end{matrix}} ) \times 100 \\$$

1. Method for selectively hydrolysing a pendant ester formed by a hydrocarbon group and a pendant carboxylate moiety in which the pendant carboxylate moiety is part of a polymer or a polymerisable compound, that comprises at least one other hydrolysable group, wherein the method comprises contacting the polymer or polymerisable compound with a hydrolytic enzyme to catalyse the hydrolysis of the pendant ester.

2. Method according to claim 1, wherein the carboxylate moiety is part of a polymer or a polymerisable compound comprising (a) at least two polymerisable moieties and (b) at least one amino acid residue.

3. Method according to claim 1 wherein the polymer or polymerisable compound comprises—in addition to the pendant ester formed by the hydrocarbon group and the pendant carboxylate moiety, a moiety selected from urea groups, thio-urea groups, urethane groups, thio-urethane groups, ester groups, amide groups, glycopeptide groups, carbonate groups, sulphones or carbohydrate groups.

4. Method according to claim 1 wherein the hydrocarbon group is an alkyl group.

5. Method according to claim 4 wherein the alkyl group is a methyl or ethyl group.

6. Method according to claim 1 wherein the polymerisable compound is represented by the formula I 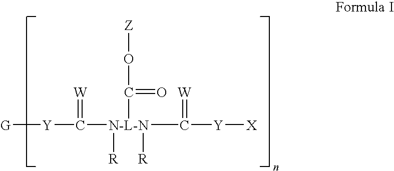
wherein
G is a residue of a polyfunctional compound having at least n functional groups or a moiety X;
each X independently represents a moiety comprising a polymerisable group;
each Y independently represents O, S or NR;
each R independently represents hydrogen or a group selected from substituted and unsubstituted hydrocarbons which optionally contain one or more heteroatoms,
L represents a substituted or unsubstituted hydrocarbon which optionally contains one or more heteroatoms,
n is an integer having a value of at least 1—W is O or S
Z is a substituted or unsubstituted hydrocarbon group bound to the carboxylate moiety.

7. Method according to claim 6 whereby
G is X;—each X-Y moiety independently represents a moiety comprising hydroxyethylacrylate or hydroxyethylmethacrylate;
each Y independently represents O;
each R independently represents hydrogen.
L represents an amino acid residue—n is an integer having a value of 1
W is O and
Z is a methyl group, ethyl group or n-propyl group bound to the carboxylate moiety.

8. Method according to claim 7 whereby the amino acid residue is in the L-configuration.

9. Method according to claim 1, wherein the polymer is a polymer composed of a compound.

10. Method according to claim 1 whereby the polymer or polymerisable compound contains one or more lysine-methylester moieties.

11. Method according to claim 1, wherein the hydrolytic enzyme is chosen from enzymes classified as hydrolases acting on ester bonds (E. C. 3.1.) or hydrolases acting on peptide bonds (E. C. 3.4.).

12. Method according to claim 1, wherein the hydrolytic enzyme is chosen from enzymes classified as serine endopeptidases (E. C. 3.4.21) or cysteine endopeptidases (E.C. 3.4.22).

13. Method according to claim 1, wherein the hydrolytic enzyme is chosen from enzymes classified as papain (E. C. 3.4.22.2) or subtilisins (E. C. 3.4.21.62).

14. Method according to claim 13 wherein the hydrolytic enzyme is Subtilisin Carlsberg®.

15. Method according to claim 1 wherein the hydrolytic enzyme classified as a hydrolase acting on a carboxylic ester bonds (E. C. 3.1.1.3) is chosen from lipozyme, lipase from Rhizomucor Miehei, lilipase, lipase B from Candida Antarctica or lipase from Penicillium Camembertii.
